(19) United States
(12) Patent Application Publication
(10) Pub. No.: US 2026/0047866 A1
(43) Pub. Date: Feb. 19, 2026

(54) OPERATION DEVICE AND IMPLANTED INDWELLING INSTRUMENT

(71) Applicant: TERUMO KABUSHIKI KAISHA, Tokyo (JP)

(72) Inventor: Masahiro ISHIDA, Oiso-machi Naka-gun (JP)

(73) Assignee: TERUMO KABUSHIKI KAISHA, Tokyo (JP)

(21) Appl. No.: 19/368,028

(22) Filed: Oct. 24, 2025

Related U.S. Application Data
(63) Continuation of application No. PCT/JP2024/015639, filed on Apr. 19, 2024.

(30) Foreign Application Priority Data
Apr. 25, 2023 (JP) 2023-071829

(51) Int. Cl. A61B 17/34

(52) U.S. Cl. CPC A61B 17/3468

(57) ABSTRACT

An operation device includes: an outer cylinder that defines a lumen penetrating inside the outer cylinder in an axial direction; and a shaft that is disposed in the lumen and is rotatable relative to the outer cylinder in a circumferential direction of the outer cylinder. The outer cylinder includes: an outer cylinder main body portion having a cylindrical shape, and one or more first holding portions that protrude from a distal end surface of the outer cylinder main body portion only in a partial region of the outer cylinder main body portion in the circumferential direction. The shaft includes: a shaft main body portion having a cylindrical shape or a columnar shape, and one or more second holding portions that protrude from a distal end surface of the shaft main body portion only in a partial region of the shaft main body portion in the circumferential direction.

### CROSS-REFERENCE TO RELATED APPLICATIONS

This application is a bypass continuation of PCT Application No. PCT/JP2024/015639, filed on April 19, 2024, which claims priority to Japanese Patent Application 2023-071829, filed on April 25, 2023. The entire contents of these disclosures are hereby incorporated by reference herein.

### BACKGROUND

The present disclosure relates to an operation device and an implant indwelling instrument.

Various therapeutic methods for indwelling an implant in a living body tissue have been proposed. For example, Japanese Patent Publication No. 2020-127607A (“PTL 1”) discloses a device that promotes regeneration of a damaged tissue by implanting porous collagen fibers having numerous pores formed therein in a damaged site.

In order to reliably indwell a string-like implant in the living body tissue, it is desirable to reliably hold and release one end of the implant. PCT Publication No. WO 2022/158406 (“PTL 2”) discloses an operation device capable of reliably holding and releasing one end of an implant and reliably indwelling the implant in a body.

The operation device disclosed in PTL 2 includes an outer cylinder, a shaft disposed in a lumen of the outer cylinder, and a gripping mechanism that is provided on the outer cylinder and the shaft and grips a string-like implant so as to be switchable between a fixed state in which the implant cannot be detached and a release state in which the implant can be detached. The gripping mechanism in PTL 2 includes a first insertion hole penetrating a side portion of the outer cylinder in a radial direction, and a second insertion hole provided in the shaft at a portion corresponding to the first insertion hole and penetrating a side portion of the shaft in a radial direction. In addition, PTL 2 discloses the first insertion hole and the second insertion hole that are formed in a groove shape and open at a distal end.

### SUMMARY

In the gripping mechanism of the operation device disclosed in PTL 2, the first insertion hole in the outer cylinder and the second insertion hole in the shaft are aligned with each other, so that the string-like implant is brought into the release state in which the implant can be detached. In the gripping mechanism of the operation device disclosed in PTL 2, the outer cylinder and the shaft are rotated relative to each other from the release state. Accordingly, a positional relationship in a circumferential direction between the first insertion hole in the outer cylinder and the second insertion hole in the shaft varies. Therefore, the string-like implant is held between the outer cylinder and the shaft, and the string-like implant is in the fixed state in which the implant cannot be detached. As described above, in the operation device disclosed in PTL 2, it is possible to switch between the fixed state in which the string-like implant cannot be detached and the release state in which the string-like implant can be detached, by rotating the outer cylinder and the shaft relative to each other.

In the operation device disclosed in PTL 2, the first insertion hole in the outer cylinder and the second insertion hole in the shaft each have a groove shape, and an insertion passage for inserting the string-like implant is provided in the groove. Therefore, according to the operation device disclosed in PTL 2, the string-like implant can be easily disposed in the insertion passage in the groove through an opening at a distal end of the groove when the gripping mechanism is in the release state described above. However, since both the first insertion hole in the outer cylinder and the second insertion hole in the shaft have a groove shape, when a relative positional relationship in the circumferential direction between the outer cylinder and the shaft in the release state is different from a desired positional relationship, a width of the insertion passage in the circumferential direction is narrowed at both end portions of the insertion passage in an extending direction, and it may be difficult to dispose the string-like implant in the insertion passage.

An object of the present disclosure is to provide an operation device and an implant indwelling instrument that can reliably grip and release a string-like implant and that can easily dispose the string-like implant in an insertion passage for the implant.

An operation device according to a first aspect of the present disclosure is (1) an operation device including: an outer cylinder that is defined with a lumen penetrating inside the outer cylinder in an axial direction; and a shaft that is disposed in the lumen and is rotatable relative to the outer cylinder in a circumferential direction of the outer cylinder, in which the outer cylinder includes an outer cylinder main body portion having a cylindrical shape, and a first holding portion that protrudes from a distal end surface of the outer cylinder main body portion only in a partial region of the outer cylinder main body portion in the circumferential direction, the shaft includes a shaft main body portion having a cylindrical shape or a columnar shape, and a second holding portion that protrudes from a distal end surface of the shaft main body portion only in a partial region of the shaft main body portion in the circumferential direction and that is configured to hold the implant disposed in an insertion passage for the implant between the first holding portion and the second holding portion by rotating the shaft relative to the outer cylinder in the circumferential direction, the insertion passage being defined between the first holding portion and the second holding portion, and at least one holding portion of the first holding portion of the outer cylinder and the second holding portion of the shaft is disposed with the number of only one in the circumferential direction.

The operation device according to an embodiment of the present disclosure is (2) the operation device according to (1), in which the first holding portion of the outer cylinder and the second holding portion of the shaft are each disposed with the number of only one in the circumferential direction.

The operation device according to an embodiment of the present disclosure is (3) the operation device according to (2), in which the insertion passage is formed by a gap that is defined between the first holding portion and the second holding portion in a state in which the first holding portion of the outer cylinder and the second holding portion of the shaft are disposed to face each other in a radial direction of the outer cylinder, the gap having a distal end open to an outside, and the gap includes an inlet space having a width gradually increasing toward the distal end.

The operation device according to an embodiment of the present disclosure is (4) the operation device according to (1), in which one holding portion of the first holding portion of the outer cylinder and the second holding portion of the shaft is disposed with the number of only one in the circumferential direction, the other holding portion is disposed with the number of two at an interval in the circumferential direction, and an insertion groove through which the implant is inserted is defined between the two other holding portions.

The operation device according to an embodiment of the present disclosure is (5) the operation device according to (4), in which the insertion passage is formed by a gap that is defined between the first holding portion and the second holding portion in a state in which the first holding portion of the outer cylinder and the second holding portion of the shaft are disposed to face each other in a radial direction of the outer cylinder, the gap having a distal end open to an outside, and the gap or the insertion groove includes an inlet space having a width gradually increasing toward the distal end.

The operation device according to an embodiment of the present disclosure is (6) the operation device according to any one of (1) to (5), in which the insertion passage is formed by a gap that is defined between the first holding portion and the second holding portion in a state in which the first holding portion of the outer cylinder and the second holding portion of the shaft are disposed to face each other in the radial direction of the outer cylinder, the gap having a distal end open to an outside, and the gap is eccentrically located outward in the radical direction relative to a central axis of the outer cylinder.

The operation device according to an embodiment of the present disclosure is (7) the operation device according to any one of (1) to (6), in which at least one holding portion of the first holding portion of the outer cylinder and the second holding portion of the shaft includes a protruding portion protruding in the circumferential direction.

An implant indwelling instrument according to a second aspect of the present disclosure is (8) an implant indwelling instrument including: the operation device according to any one of (1) to (7); and a puncture needle including an outer needle into which the outer cylinder and the shaft of the operation device are insertable.

### Advantageous Effect of Invention

According to the present disclosure, it is possible to provide the operation device and the implant indwelling instrument that can reliably grip and release the string-like implant and that can easily dispose the string-like implant in the insertion passage for the implant.

### BRIEF DESCRIPTION OF THE DRAWINGS

FIG. 1 is a view illustrating an implant indwelling instrument according to an embodiment of the present disclosure.

FIG. 2 is a view illustrating an operation device according to an embodiment of the present disclosure.

FIG. 3 is a view illustrating a usage example of the implant indwelling instrument illustrated in FIG. 1, and is a view illustrating a state in which an outer needle and an inner needle of a puncture needle puncture a target site in a living body from a living body surface.

FIG. 4 is a view illustrating a usage example of the implant indwelling instrument illustrated in FIG. 1, and is a view illustrating a state before the operation device is inserted into an outer needle assembly of the puncture needle.

FIG. 5 is a view illustrating a usage example of the implant indwelling instrument illustrated in FIG. 1, and is a view illustrating a state in which the operation device is inserted into the outer needle assembly of the puncture needle.

FIG. 6 is a view illustrating a usage example of the implant indwelling instrument illustrated in FIG. 1, and is a view illustrating a state in which the operation device and the outer needle assembly of the puncture needle 10 are removed from the living body after an implant is indwelled in the target site in the living body.

FIG. 7A is a perspective view illustrating a distal portion of an outer cylinder of the operation device illustrated in FIG. 2.

FIG. 7B is a side view illustrating the distal portion of the outer cylinder of the operation device illustrated in FIG. 2.

FIG. 8A is a perspective view illustrating a distal portion of a shaft of the operation device illustrated in FIG. 2.

FIG. 8B is a side view illustrating the distal portion of the shaft of the operation device illustrated in FIG. 2.

FIG. 9A is a perspective view illustrating a state in which a string-like implant is disposed in an insertion passage for the string-like implant, the insertion passage being defined between a first holding portion of the outer cylinder and a second holding portion of the shaft.

FIG. 9B is a side view illustrating the state illustrated in FIG. 9A.

FIG. 9C is a cross-sectional view taken along a line I-I in FIG. 9B.

FIG. 10 is a cross-sectional view at the same position as in FIG. 9C, and is a view illustrating a state in which the shaft is rotated relative to the outer cylinder from the state in FIG. 9C, and the string-like implant is gripped by being held between the first holding portion and the second holding portion.

FIG. 11 is a view illustrating a modification of the outer cylinder and the shaft.

FIG. 12 is a view illustrating a modification of the outer cylinder and the shaft.

FIG. 13 is a view illustrating a modification of a gap for forming the insertion passage for the string-like implant.

FIG. 14A is a view illustrating an example in which an inlet space is formed in the configuration illustrated in FIG. 11.

FIG. 14B is a view illustrating an example in which an inlet space is formed in the configuration illustrated in FIG. 12.

FIG. 15A illustrates a modification of a formation position of the insertion passage.

FIG. 15B illustrates a modification of a formation position of the insertion passage.

FIG. 16A is a view illustrating a second holding portion including a protruding portion as a modification of the second holding portion, and is a view illustrating a case where the gripping mechanism is in a release state.

FIG. 16B is a view illustrating the second holding portion including the protruding portion in FIG. 16A, and is a view illustrating a case where the gripping mechanism is in a gripping state.

FIG. 17A is a view illustrating a first holding portion including a protruding portion as a modification of the first holding portion.

FIG. 17B is a view illustrating a first holding portion and a second holding portion each including a protruding portion as a modification of the first holding portion and the second holding portion.

FIG. 18A is a view illustrating an example of a method for manufacturing only one first holding portion provided in a circumferential direction.

FIG. 18B is a view illustrating an example of a method for manufacturing two first holding portions provided at an interval in a circumferential direction.

FIG. 19 is a view illustrating a first holding portion of an outer cylinder and a second holding portion of a shaft as a comparative example.

### DETAILED DESCRIPTION

Hereinafter, embodiments of an operation device and an implant indwelling instrument according to the present disclosure will be exemplified and described with reference to the drawings. In the drawings, the same components are denoted by the same reference numerals.

Hereinafter, unless otherwise specified, a side near a user who uses the operation device and the implant indwelling instrument and performs an operation in a state in which the operation device and the implant indwelling instrument according to the present disclosure are inserted into a living body is referred to as a "proximal side" of the operation device and the implant indwelling instrument, and a side away from the user is referred to as a "distal side".

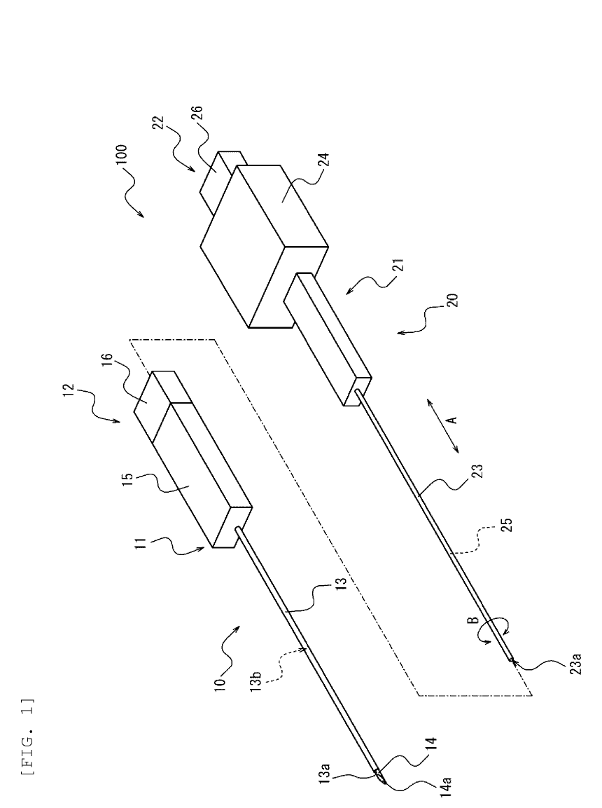

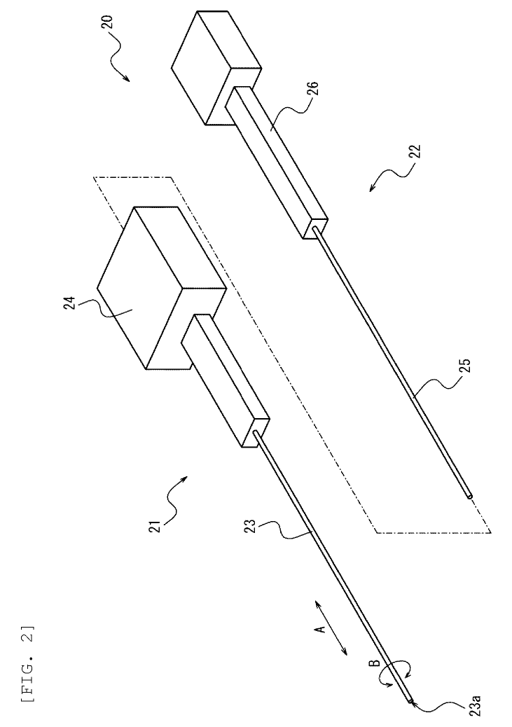

FIG. 1 is a view illustrating an implant indwelling instrument 100 as an embodiment of the implant indwelling instrument according to the present disclosure. As illustrated in FIG. 1, the implant indwelling instrument 100 includes a puncture needle 10 and an operation device 20 as an embodiment of the operation device according to the present disclosure. The puncture needle 10 is provided with an outer needle assembly 11 including an outer needle 13, and an inner needle assembly 12 including an inner needle 14. FIG. 2 is a view illustrating the operation device 20. As illustrated in FIG. 2, the operation device 20 includes an outer cylinder assembly 21 and a shaft assembly 22.

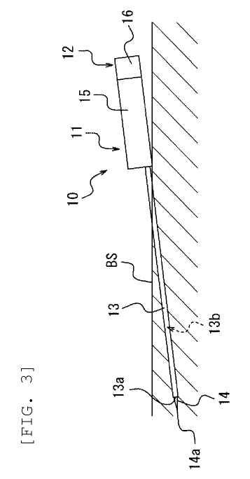

FIGS. 3 to 6 are views illustrating usage examples of the implant indwelling instrument 100. First, a usage example of the implant indwelling instrument 100 will be described with reference to FIGS. 3 to 6.

First, the puncture needle 10 punctures a living body from a living body surface BS. FIG. 3 illustrates a state in which the outer needle 13 and the inner needle 14 of the puncture needle 10 puncture a target site in the living body from the living body surface BS. A sharp needle distal end is formed at a distal end 14a of the inner needle 14 of the puncture needle 10. The inner needle assembly 12 is disposed in the outer needle assembly 11 such that the distal end 14a of the inner needle 14 protrudes from a distal end 13a of the outer needle 13. In this state, the outer needle 13 and the inner needle 14 of the puncture needle 10 puncture the living body from the living body surface BS toward the target site in the living body. After the outer needle 13 and the inner needle 14 of the puncture needle 10 puncture the target site, the inner needle assembly 12 including the inner needle 14 is removed from the living body. Accordingly, the outer needle assembly 11 in a state in which the outer needle 13 punctures the living body from the living body surface BS can be indwelled.

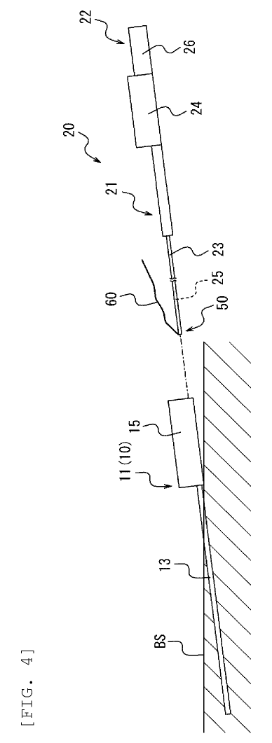

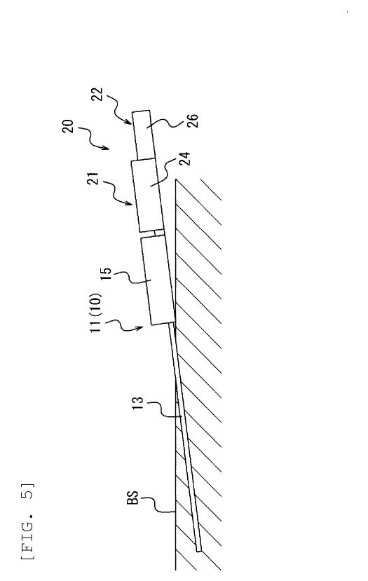

Next, the operation device 20 is inserted into the indwelled outer needle assembly 11 of the puncture needle 10. FIG. 4 illustrates a state before the operation device 20 is inserted into the outer needle assembly 11 of the puncture needle 10. FIG. 5 illustrates a state in which the operation device 20 is inserted into the outer needle assembly 11 of the puncture needle 10. As illustrated in FIG. 4, the operation device 20 includes a gripping mechanism 50 that grips a string-like implant 60 by holding the implant 60 at a distal portion. The gripping mechanism 50 is implemented by the outer cylinder assembly 21 and the shaft assembly 22. The gripping mechanism 50 can switch a state between a gripping state in which the string-like implant 60 is held and undetachable and a release state in which the implant 60 is not held and is detachable. The details will be described later. By using the gripping mechanism 50, the operation device 20 can be inserted into the outer needle assembly 11 of the puncture needle 10 in a state in which the implant 60 is gripped at the distal portion. In this manner, the implant 60 can be conveyed to a target site in the living body through the outer needle 13 of the outer needle assembly 11 by using the operation device 20.

Next, the implant 60 is indwelled at the target site in the living body. After the implant 60 is indwelled at the target site in the living body, the operation device 20 is removed from the living body together with the outer needle assembly 11 of the puncture needle 10. FIG. 6 illustrates a state in which the operation device 20 and the outer needle assembly 11 of the puncture needle 10 are removed from the living body after the implant 60 is indwelled at the target site in the living body. The implant 60 is indwelled at the target site in the living body by releasing the gripping state of the gripping mechanism 50 in the operation device 20. In this manner, the implant 60 can be indwelled at the target site in the living body by using the operation device 20.

In FIGS. 3 to 6, a technique according to a so-called "non-penetration method" is described in which the implant 60 is indwelled at the target site in the living body in a state in which the distal end 13a of the outer needle 13 of the puncture needle 10 is positioned in the living body, but the operation device 20 and the implant indwelling instrument 100 including the operation device 20 are not limited to the application to the technique according to the non-penetration method. The operation device 20 and the implant indwelling instrument 100 including the operation device 20 may be used, for example, for a technique according to a so-called "penetration method" in which the implant 60 is indwelled at a target site in a living body in a state in which the outer needle 13 of the puncture needle 10 penetrates the living body and the distal end 13a of the outer needle 13 is located outside the living body.

Hereinafter, the implant indwelling instrument 100 will be disclosed in detail.

### <Puncture Needle 10>

As described above, the puncture needle 10 includes the outer needle assembly 11 and the inner needle assembly 12. The puncture needle 10 is assembled by disposing the inner needle assembly 12 inside the outer needle assembly 11. The inner needle assembly 12 is detachable from the outer needle assembly 11 from a proximal side of the outer needle assembly 11 to an inner side of the outer needle assembly 11.

The outer needle assembly 11 includes the cylindrical outer needle 13 and an outer needle hub 15 coupled to a proximal portion of the outer needle 13. The distal end 13a of the outer needle 13 is not provided with a sharp needle distal end. A lumen 13b extending through the outer needle 13 in an axial direction is defined inside the outer needle 13. The outer needle hub 15 defines a hollow portion communicating with the lumen 13b of the outer needle 13.

The inner needle assembly 12 includes the inner needle 14 having the sharp distal end 14a and an inner needle hub 16 coupled to a proximal side of the inner needle 14. The inner needle 14 is disposed in the lumen 13b of the outer needle 13 so as to be movable in the axial direction. The sharp distal end 14a of the inner needle 14 can be allowed to protrude from the distal end 13a of the outer needle 13 by pushing the inner needle hub 16 to a distal side in the axial direction with respect to the outer needle assembly 11. As described above, in this state, the puncture needle 10 punctures the target site in the living body such as a patient from the living body surface BS of the living body (see FIG. 3 and the like).

### <Operation Device 20>

As illustrated in FIG. 2, the operation device 20 is assembled by arranging the shaft assembly 22 inside the outer cylinder assembly 21.

The outer cylinder assembly 21 includes an elongated outer cylinder 23 and an outer cylinder hub 24 coupled to a proximal side of the outer cylinder 23. The outer cylinder 23 is a cylindrical member made of metal such as stainless steel or hard resin. A lumen 23a extending through the outer cylinder 23 in an axial direction A along a central axis of the outer cylinder 23 is defined inside the outer cylinder 23. The outer cylinder 23 is formed to have an outer diameter at which the outer cylinder 23 can be inserted into the outer needle 13 of the puncture needle 10 illustrated in FIG. 1. The lumen 23a of the outer cylinder 23 is open to the proximal side and communicates with a hollow portion of the outer cylinder hub 24. The outer cylinder hub 24 may be made of, for example, a hard resin material such as polycarbonate resin.

The shaft assembly 22 includes a shaft 25 and a shaft hub 26 coupled to a proximal portion of the shaft 25. The shaft 25 in the present embodiment is a columnar solid member having an outer diameter at which the shaft 25 can be inserted into the lumen 23a of the outer cylinder 23. Alternatively, the shaft 25 may have a cylindrical shape. The shaft 25 may be made of, for example, metal such as stainless steel. The shaft hub 26 may be made of, for example, a hard resin material such as polycarbonate resin.

The shaft 25 is disposed in the lumen 23a of the outer cylinder 23. That is, the shaft assembly 22 can be attached to the outer cylinder assembly 21 so that the shaft 25 is inserted into the lumen 23a of the outer cylinder 23. In a state in which the shaft assembly 22 is attached to the outer cylinder assembly 21, the shaft assembly 22 is rotatable relative to the outer cylinder assembly 21 in a circumferential direction B around the central axis of the outer cylinder 23. That is, the shaft 25 is rotatable relative to the outer cylinder 23 in the circumferential direction B of the outer cylinder 23 in a state in which the shaft 25 is disposed in the lumen 23a of the outer cylinder 23.

### <<Gripping Mechanism 50>>

Next, the gripping mechanism 50 of the operation device 20 will be described. The gripping mechanism 50 is implemented by the outer cylinder assembly 21 and the shaft assembly 22 described above.

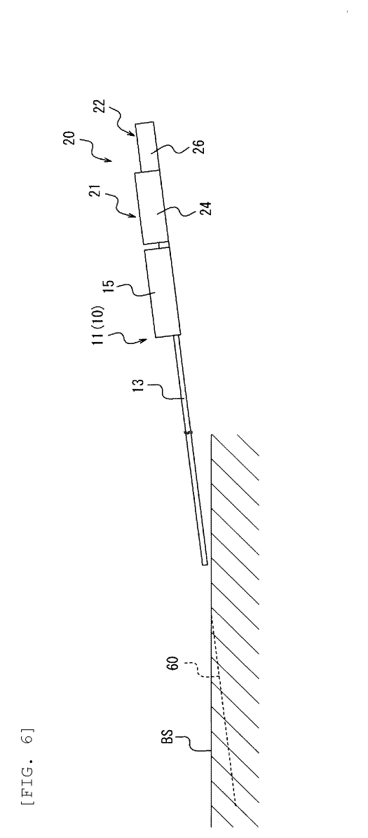

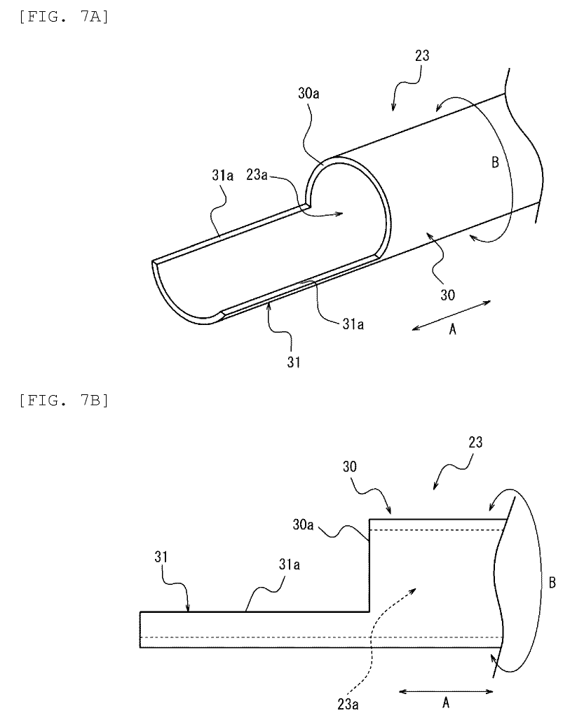

FIG. 7A is a perspective view illustrating a distal portion of the outer cylinder 23. FIG. 7B is a side view illustrating the distal portion of the outer cylinder 23. As illustrated in FIGS. 7A and 7B, a part of a cylindrical peripheral wall in the circumferential direction B is cut out at the distal portion of the outer cylinder 23.

The gripping mechanism 50 uses a distal end protruding portion formed by cutting out the distal portion of the outer cylinder 23 illustrated in FIGS. 7A and 7B to hold the string-like implant 60 (see FIG. 4 and the like) with the shaft 25.

As described above, the outer cylinder 23 includes a cylindrical outer cylinder main body portion 30 and a first holding portion 31 protruding from a distal end surface 30a of the outer cylinder main body portion 30 only in a partial region of the outer cylinder main body portion 30 in the circumferential direction B.

A proximal end of the outer cylinder main body portion 30 is coupled to the outer cylinder hub 24 described above. The first holding portion 31 in the present embodiment is formed by extending only a partial region of the peripheral wall of the outer cylinder main body portion 30 in the circumferential direction B in a manner of protruding from the distal end surface 30a of the outer cylinder main body portion 30 to the distal side. Therefore, the first holding portion 31 in the present embodiment is implemented by a curved plate portion that is curved in an arc shape in a cross-sectional view orthogonal to the axial direction A.

As illustrated in FIGS. 7A and 7B, the distal end surface 30a of the outer cylinder main body portion 30 in the present embodiment is a surface substantially orthogonal to the axial direction A, but the disclosure is not limited to this configuration. The distal end surface 30a may be, for example, a surface inclined relative to the axial direction A.

End surfaces 31a on both sides of the first holding portion 31 in the circumferential direction B in the present embodiment are surfaces substantially parallel to the axial direction A, but the disclosure is not limited to this configuration. The end surface 31a may extend, for example, in a direction inclined relative to the axial direction A.

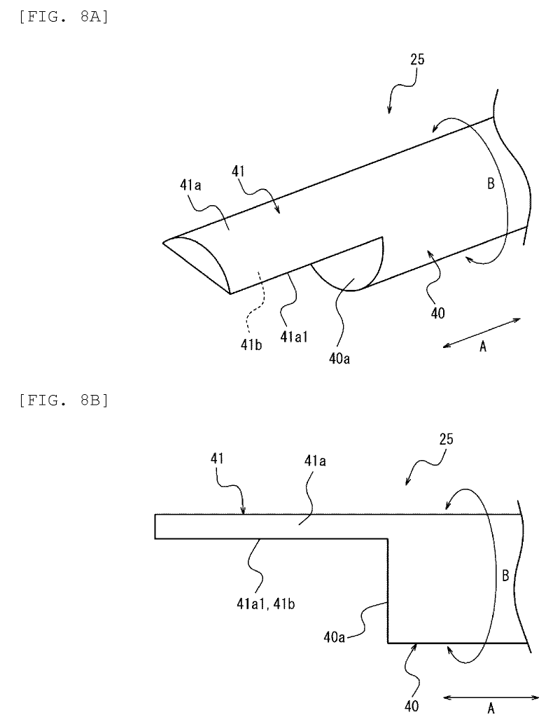

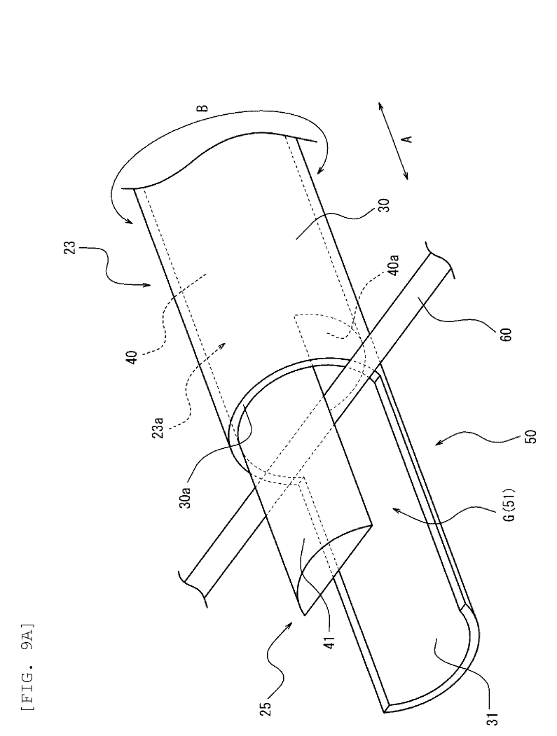

FIG. 8A is a perspective view illustrating a distal portion of the shaft 25. FIG. 8B is a side view illustrating the distal portion of the shaft 25. Hereinafter, the shaft 25 alone will be described using the "axial direction A" and the "circumferential direction B" of the outer cylinder 23 in a state in which the shaft 25 is disposed in the outer cylinder 23. As illustrated in FIGS. 8A and 8B, a part of a columnar portion of the shaft 25 in the circumferential direction B is cut out at the distal portion of the shaft 25. The gripping mechanism 50 uses a distal end protruding portion formed by cutting out the distal portion of the shaft 25 illustrated in FIGS. 8A and 8B to hold the string-like implant 60 (see Fig.4 and the like) with the outer cylinder 23.

As described above, the shaft 25 includes a columnar shaft main body portion 40 and a second holding portion 41 protruding from a distal end surface 40a of the shaft main body portion 40 only in a partial region of the shaft main body portion 40 in the circumferential direction B. As will be disclosed in detail later, by rotating the shaft 25 relative to the outer cylinder 23 in the circumferential direction B, the implant 60 disposed in an insertion passage 51 for the string-like implant 60, the insertion passage 51 being defined between the second holding portion 41 and the first holding portion 31, can be held between the second holding portion 41 and the first holding portion 31.

A proximal end of the shaft main body portion 40 is coupled to the shaft hub 26 described above. The second holding portion 41 in the present embodiment is formed by extending a portion including only a partial region in the circumferential direction B of an outer peripheral surface of the shaft main body portion 40 in a manner of protruding from the distal end surface 40a of the shaft main body portion 40 to a distal side. Therefore, the second holding portion 41 in the present embodiment is implemented by a rod-shaped protruding portion having an outer surface 41a that is curved in an arc shape in a cross-sectional view orthogonal to the axial direction A.

As illustrated in FIGS. 8A and 8B, the distal end surface 40a of the shaft main body portion 40 in the present embodiment is a surface substantially orthogonal to the axial direction A, but the disclosure is not limited to this configuration. The distal end surface 40a may be, for example, a surface inclined relative to the axial direction A.

An inner surface 41b of the rod-shaped protruding portion serving as the second holding portion 41 in the present embodiment is a surface substantially parallel to the axial direction A, but the disclosure is not limited to this configuration. The inner surface 41b may be, for example, a concave surface or a convex surface. Further, the outer surface 41a in the present embodiment intersects the inner surface 41b at outer edge portions 41a1 at both ends in the circumferential direction B. The outer edge portion 41a1 is implemented by a ridge portion extending substantially parallel to the axial direction A, but the disclosure is not limited to this configuration. The ridge portion serving as the outer edge portion 41a1 may extend, for example, in a direction inclined relative to the axial direction A.

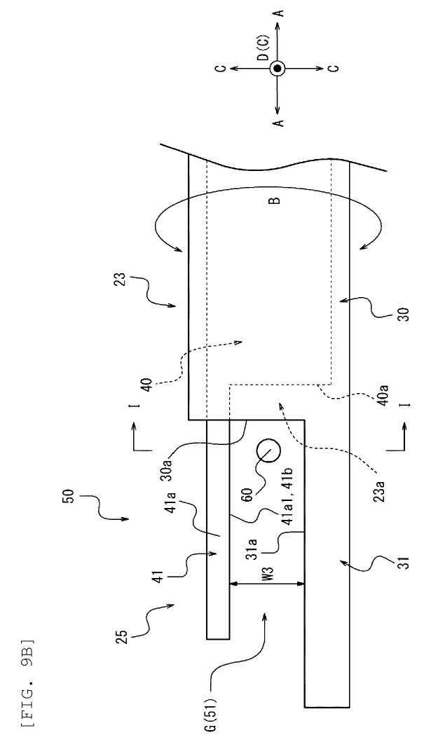

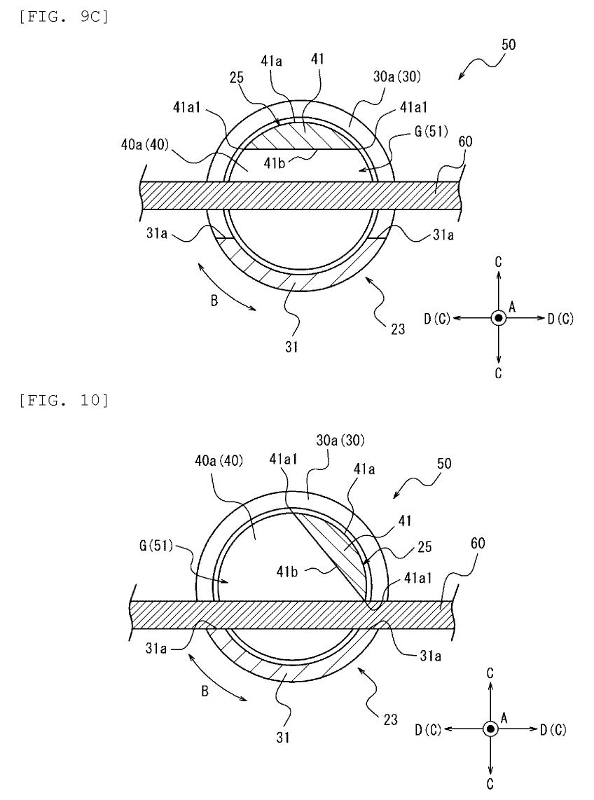

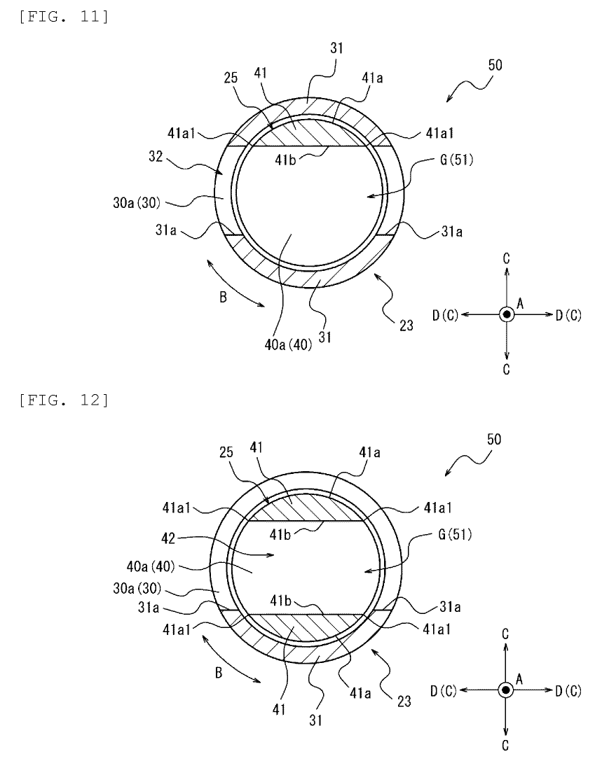

Next, a gripping operation and a gripping release operation on the string-like implant 60 by the first holding portion 31 and the second holding portion 41 described above will be described with reference to FIG. 9A to FIG. 9C and FIG. 10. FIG. 9A is a perspective view illustrating a state in which the string-like implant 60 is disposed in the insertion passage 51 for the string-like implant 60, the insertion passage 51 being defined between the first holding portion 31 and the second holding portion 41, in a state in which the shaft 25 is disposed in the outer cylinder 23. FIG. 9B is a side view illustrating the state illustrated in FIG. 9A. FIG. 9C is a cross-sectional view taken along a line I-I in FIG. 9B. FIGS. 9A to 9C illustrate a release state of the gripping mechanism 50. FIG. 10 is a cross-sectional view at the same position as FIG. 9C, and is a view illustrating a state in which the shaft 25 is rotated relative to the outer cylinder 23 from the state in FIG. 9C and the string-like implant 60 is held by being interposed between the first holding portion 31 and the second holding portion 41. That is, FIG. 10 illustrates a gripping state of the gripping mechanism 50.

FIGS. 9A to 9C illustrate a state in which the shaft 25 is disposed in the outer cylinder 23, and the first holding portion 31 of the outer cylinder 23 and the second holding portion 41 of the shaft 25 are disposed to face each other in a radial direction C of the outer cylinder 23 (see FIGS. 9B and 9C). As described above, the states illustrated in FIGS. 9A to 9C are the release state of the gripping mechanism 50 in the present embodiment. In the release state, the insertion passage 51 for the string-like implant 60 is formed by a gap G that is defined between the first holding portion 31 and the second holding portion 41 and has a distal end open to the outside. In the release state of the gripping mechanism 50, a minimum width of the gap G is larger than a diameter of the string-like implant 60. Accordingly, the string-like implant 60 can be disposed in the gap G from the open end on a distal side of the gap G for forming the insertion passage 51.

Thereafter, when the shaft 25 is rotated relative to the outer cylinder 23 in one direction of the circumferential direction B, the minimum width of the gap G for forming the insertion passage 51 is reduced. Accordingly, the minimum width of the gap G can be made equal to or smaller than the diameter of the string-like implant 60. Therefore, as illustrated in FIG. 10, the string-like implant 60 is held between the first holding portion 31 of the outer cylinder 23 and the second holding portion 41 of the shaft 25. More specifically, the implant 60 is held by being interposed in the circumferential direction B between the end surface 31a of the first holding portion 31 and the outer edge portion 41a1 of the outer surface 41a of the second holding portion 41. Accordingly, the gripping mechanism 50 is brought into the gripping state in which the implant 60 is held and undetachable. In the gripping state, the implant 60 is conveyed to a target site in a living body (see FIGS. 4 and 5). That is, the implant 60 can be conveyed to a target site in a living body by inserting the outer cylinder 23 and the shaft 25 that grip the implant 60 at distal end portions into the outer needle 13 of the puncture needle 10 (see FIG. 4 and the like).

From the gripping state of the gripping mechanism 50 illustrated in FIG. 10, when the shaft 25 is rotated relative to the outer cylinder 23 so as to return in an opposite direction opposite to the one direction of the circumferential direction B, the gripping mechanism 50 returns to the state illustrated in FIGS. 9A to 9C. Accordingly, a state in which the string-like implant 60 is held between the first holding portion 31 of the outer cylinder 23 and the second holding portion 41 of the shaft 25 can be released. That is, the gripping mechanism 50 returns to the release state in which the implant 60 can be detached. The implant 60 can be indwelled at the target site in the living body (see FIG. 6) by changing the gripping mechanism 50 from the gripping state to the release state.

As described above, the gripping state and the release state of the gripping mechanism 50 can be switched by rotating the outer cylinder 23 and the shaft 25 relative to each other.

Here, in the present embodiment, only one first holding portion 31 of the outer cylinder 23 and only one second holding portion 41 of the shaft 25 are disposed in the circumferential direction B. Specifically, only one curved plate portion serving as the first holding portion 31 in the present embodiment is provided in the circumferential direction B. In addition, only one rod-shaped protruding portion serving as the second holding portion 41 in the present embodiment is provided in the circumferential direction B. With such a configuration, in the release state illustrated in FIG. 9C, even when a positional relationship between the outer cylinder 23 and the shaft 25 in the circumferential direction B is slightly deviated from a desired positional relationship, the string-like implant 60 is easily disposed in the insertion passage 51. Hereinafter, this point will be described using a comparative example.

FIG. 19 is a view illustrating a gripping mechanism 550 as a comparative example. In an outer cylinder 523 in FIG. 19, two first holding portions 531 are disposed at an interval in the circumferential direction B. The two first holding portions 531 in FIG. 19 are arranged to face each other in the radial direction C, and a first groove 570 penetrating in a direction orthogonal to a facing direction of the two first holding portions 531 is defined between the two first holding portions 531. In a shaft 525 in FIG. 19, two second holding portions 541 are disposed at an interval in the circumferential direction B. The two second holding portions 541 in FIG. 19 are arranged to face each other in the radial direction C, and a second groove 571 penetrating in a direction orthogonal to a facing direction of the two second holding portions 541 is defined between the two second holding portions 541. FIG. 19 illustrates a release state of the gripping mechanism 550. In FIG. 19, a circumferential width at both ends of the first groove 570 of the outer cylinder 523 is equal to a circumferential width at both ends of the second groove 571 of the shaft 525. Therefore, in the release state of the gripping mechanism 550, a maximum value of a circumferential width W1 at both ends of an insertion passage 551 for the implant 60 is a circumferential width W2 at both ends of the first groove 570 and at both ends of the second groove 571. However, as illustrated in FIG. 19, in the release state of the gripping mechanism 550, a positional relationship between the outer cylinder 523 and the shaft 525 in the circumferential direction B may slightly deviate from a desired positional relationship. In such a case, even when the gripping mechanism 550 is in the release state, the circumferential width W1 at both ends of the insertion passage 551 of the implant 60 is narrower than the circumferential width W2. That is, the circumferential width W1 at both ends of the insertion passage 551 is narrowed, and it is difficult to dispose the implant 60 in the insertion passage 551.

On the other hand, only one first holding portion 31 of the outer cylinder 23 and only one second holding portion 41 of the shaft 25 are disposed in the circumferential direction B. Therefore, as in the state illustrated in FIG. 19, even when the positional relationship between the outer cylinder 23 and the shaft 25 in the circumferential direction B in the present embodiment is slightly deviated from the desired positional relationship, the circumferential width W1 at one end of the insertion passage 51 can be maintained without being narrowed. Therefore, in the release state illustrated in FIGS. 9A to 9C, even when the positional relationship between the outer cylinder 23 and the shaft 25 in the circumferential direction B is slightly deviated from the desired positional relationship, the string-like implant 60 is easily disposed in the insertion passage 51.

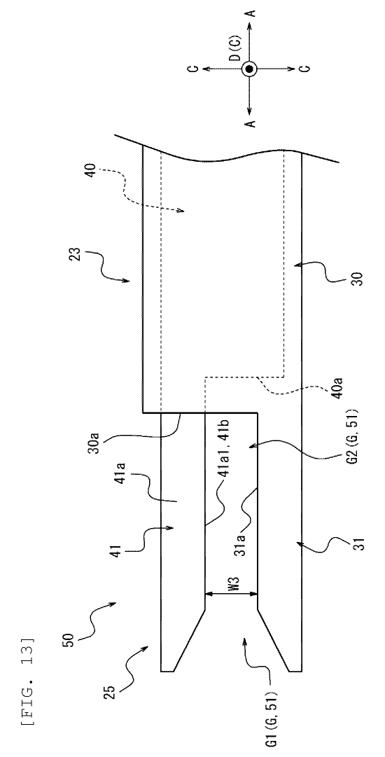

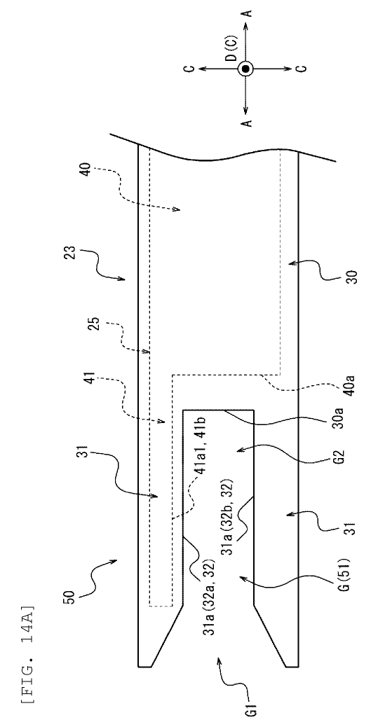

In the present embodiment, only one first holding portion 31 of the outer cylinder 23 and only one second holding portion 41 of the shaft 25 are disposed in the circumferential direction B, but the disclosure is not limited to this configuration. As illustrated in FIG. 11, only one second holding portion 41 of the shaft 25 may be disposed in the circumferential direction B, and two first holding portions 31 of the outer cylinder 23 may be disposed at an interval in the circumferential direction B. In such a case, an insertion groove 32 through which the implant 60 is inserted is defined between the two first holding portions 31 of the outer cylinder 23. As illustrated in FIG. 12, only one first holding portion 31 of the outer cylinder 23 may be disposed in the circumferential direction B, and two second holding portions 41 of the shaft 25 may be disposed at an interval in the circumferential direction B. In such a case, an insertion groove 42 through which the implant 60 is inserted is defined between the two second holding portions 41 of the shaft 25. That is, at least one of the first holding portion 31 of the outer cylinder 23 and the second holding portion 41 of the shaft 25 may be disposed with the number of only one in the circumferential direction B.

Next, a shape of the gap G for forming the insertion passage 51 in the gripping mechanism 50 in the release state will be described. As illustrated in FIGS. 9A to 9C, the gap G for forming the insertion passage 51 in the present embodiment penetrates in the radial direction C. In the radial direction C, a direction through which the gap G penetrates is defined as a penetration direction D. In such a case, FIG. 9B is a side view illustrating the outer cylinder 23 and the shaft 25 when the gap G for forming the insertion passage 51 is viewed along the penetration direction D. In the side view illustrated in FIG. 9B, a width W3 of the gap G in the present embodiment is substantially constant regardless of a position in the axial direction A, but the disclosure is not limited to this configuration. FIG. 13 is a view illustrating a modification of the gap G for forming the insertion passage 51. Similar to FIG. 9B, FIG. 13 is a side view illustrating the outer cylinder 23 and the shaft 25 when the gap G for forming the insertion passage 51 is viewed along the penetration direction D. As illustrated in FIG. 13, the gap G may include an inlet space G1 whose width W3 gradually increases toward a distal end. More specifically, in the side view illustrated in FIG. 13, the gap G is defined between the end surface 31a of the first holding portion 31 and the outer edge portion 41a1 and the inner surface 41b of the second holding portion 41. The gap G illustrated in FIG. 13 has the constant width W3 regardless of a position in the axial direction A, and includes a holding space G2 capable of holding the string-like implant 60 (see FIG. 9B and the like) and the inlet space G1 coupled to a distal side of the holding space G2 and having the width W3 gradually increasing toward the distal end. In the holding space G2, the end surface 31a of the first holding portion 31 and the outer edge portion 41a1, the inner surface 41b of the second holding portion 41 extend along the axial direction A, and a facing distance between the end surface 31a and the outer edge portion 41a1, the inner surface 41b is constant regardless of a position in the axial direction A. On the other hand, in the inlet space G1, the end surface 31a of the first holding portion 31 and the outer edge portion 41a1, the inner surface 41b of the second holding portion 41 are inclined relative to the axial direction A such that the facing distance between the end surface 31a and the outer edge portion 41a1, the inner surface 41b increases toward the distal side in the axial direction A. The string-like implant 60 can be easily disposed in the holding space G2 through the inlet space G1 in the release state by providing such an inlet space G1 in the gripping mechanism 50.

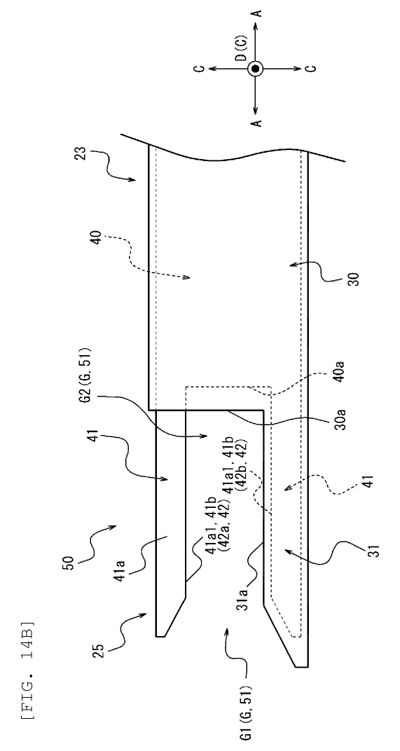

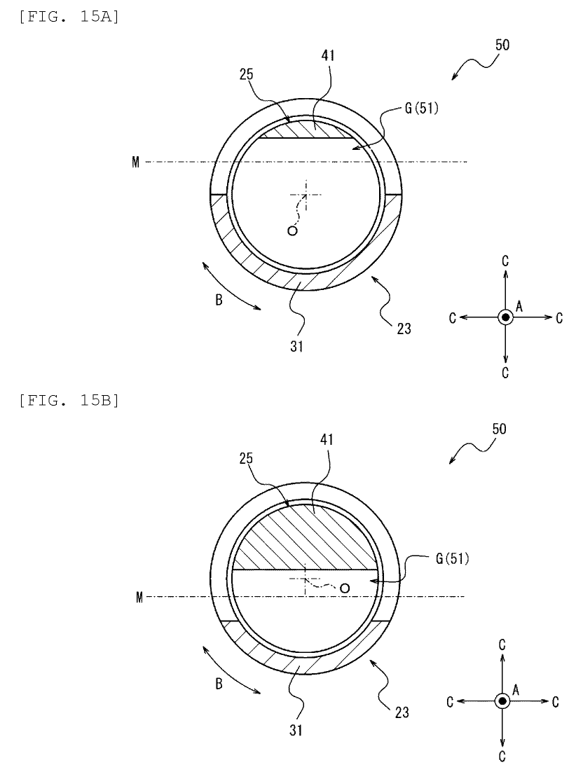

FIG. 13 illustrates a configuration in which only one first holding portion 31 and only one second holding portion 41 are disposed in a partial region in the circumferential direction B, but the same inlet space G1 and the same holding space G2 can be formed even in the configuration illustrated in FIGS. 11 and 12 described above. FIG. 14A illustrates an example in which the inlet space G1 is formed in the configuration illustrated in FIG. 11. FIG. 14B illustrates an example in which the inlet space G1 is formed in the configuration illustrated in FIG. 12.

Here, as illustrated in FIG. 14A, groove walls 32a and 32b on both sides of the insertion groove 32 defined between the two first holding portions 31 disposed at an interval in the circumferential direction B may have a width narrower than the gap G (in FIG. 14A, a gap between one groove wall 32b of the insertion groove 32 and the outer edge portion 41a1, the inner surface 41b of the second holding portion 41) for forming the insertion passage 51 in the gripping mechanism 50 in the release state. The groove walls 32a and 32b are formed by the end surface 31a of the first holding portion 31. In such a case, as illustrated in FIG. 14A, the insertion groove 32 includes the inlet space G1 whose width gradually increases toward a distal end, and the holding space G2 having a constant width regardless of a position in the axial direction A. That is, the inlet space G1 and the holding space G2 similar to those in FIG. 13 may be formed according to shapes of the groove walls 32a and 32b on both sides of the insertion groove 32. Different from the example illustrated in FIG. 14B, groove walls 42a and 42b on both sides of the insertion groove 42 defined between the two second holding portions 41 disposed at an interval in the circumferential direction B may have a width narrower than the gap G for forming the insertion passage 51 in the gripping mechanism 50 in the release state. In such a case, the insertion groove 42 includes the inlet space G1 whose width gradually increases toward a distal end, and the holding space G2 having a constant width regardless of a position in the axial direction A. That is, the inlet space G1 and the holding space G2 similar to those in FIG. 13 may be formed according to shapes of the groove walls 42a and 42b on both sides of the insertion groove 42. In this manner, when two first holding portions 31 are disposed at an interval in the circumferential direction B, the inlet space G1 may be formed in the gap G for forming the insertion passage 51 defined between the first holding portion 31 and the second holding portion 41, or in the insertion groove 32. When two second holding portions 41 are disposed at an interval in the circumferential direction B, the inlet space G1 may be formed in the gap G for forming the insertion passage 51 defined between the first holding portion 31 and the second holding portion 41, or in the insertion groove 42.

The holding space G2 illustrated in FIGS. 13 to 14B has a constant width regardless of a position in the axial direction A, but the disclosure is not limited to this shape. The holding space G2 may have, for example, a shape in which the width gradually decreases from a proximal side toward a distal side in the axial direction A.

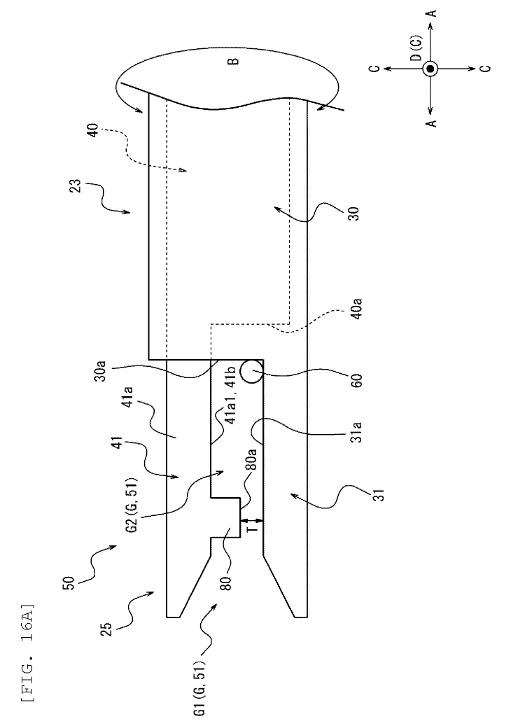

Next, a modification in which the insertion passage 51 is disposed outward in the radial direction C relative to a central axis O of the outer cylinder 23 will be described with reference to FIG. 15A and FIG. 15B. FIG. 15A illustrates an example in which a region of the first holding portion 31 in the circumferential direction B is wider than that in the configuration illustrated in FIG. 9B, and a region of the second holding portion 41 in the circumferential direction B is narrower than that in the configuration illustrated in FIG. 9B. FIG. 15B illustrates an example in which a region of the first holding portion 31 in the circumferential direction B is narrower than that in the configuration illustrated in FIG. 9B, and a region of the second holding portion 41 in the circumferential direction B is wider than that in the configuration illustrated in FIG. 9B.

FIGS. 15A and 15B illustrate a state in which the first holding portion 31 of the outer cylinder 23 and the second holding portion 41 of the shaft 25 are disposed to face each other in the radial direction C of the outer cylinder 23, that is, a release state of the gripping mechanism 50. When the gripping mechanism 50 is in the release state, the insertion passage 51 illustrated in FIGS. 15A and 15B is formed by the gap G that is defined between the first holding portion 31 and the second holding portion 41 and has a distal end open to the outside. The gap G illustrated in FIGS. 15A and 15B is disposed outward in the radial direction C relative to the central axis O of the outer cylinder 23. More specifically, the gap G illustrated in FIGS. 15A and 15B is disposed outward in the radial direction C relative to the central axis O such that a center line M of the gap G does not pass through the central axis O. In this manner, a large extension region in the circumferential direction B of one holding portion of the first holding portion 31 and the second holding portion 41 can be ensured. Therefore, by increasing the extension region in the circumferential direction B of one holding portion of the first holding portion 31 and the second holding portion 41 for which strength reinforcement is particularly desirable, bending or breakage of the one holding portion can be prevented. In particular, the first holding portion 31 of the outer cylinder 23 preferably has higher strength than the second holding portion 41 of the shaft 25. Therefore, as illustrated in FIG. 15A, it is particularly preferable to unevenly distribute the gap G such that an extension region of the first holding portion 31 of the outer cylinder 23 in the circumferential direction B is larger than an extension region of the second holding portion 41 of the shaft 25 in the circumferential direction B.

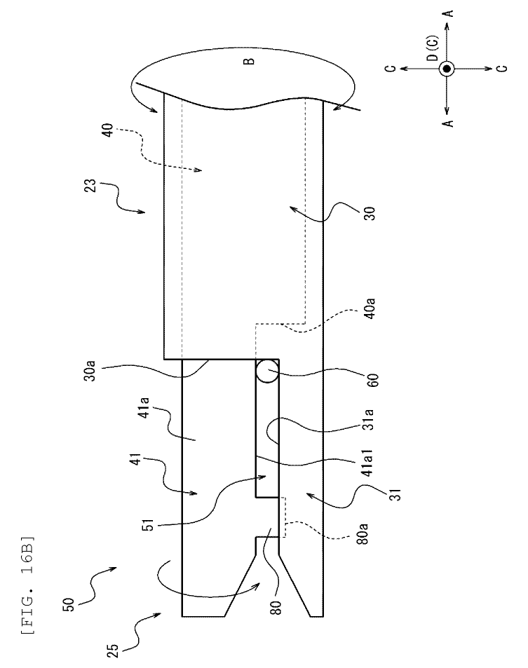

Next, a protruding portion 80 provided on at least one of the first holding portion 31 of the outer cylinder 23 and the second holding portion 41 of the shaft 25 and protruding in the circumferential direction B will be described with reference to FIGS. 16A and 16B. FIGS. 16A and 16B illustrate a configuration in which the second holding portion 41 illustrated in FIG. 13 further includes the protruding portion 80 protruding in the circumferential direction B. FIG. 16A illustrates a case where the gripping mechanism 50 is in the release state, and FIG. 16B illustrates a case where the gripping mechanism 50 is in the gripping state.

The second holding portion 41 illustrated in FIGS. 16A and 16B has the similar configuration to that illustrated in FIG. 13 except for whether the protruding portion 80 is provided. Therefore, as illustrated in FIG. 16A, the gap G for forming the insertion passage 51 includes the guide space G1 and the holding space G2 which are the same as those in the configuration illustrated in FIG. 13.

The second holding portion 41 illustrated in FIGS. 16A and 16B includes the protruding portion 80 protruding outward in the circumferential direction B from the outer edge portions 41a1 on both sides in the circumferential direction B of the outer surface 41a. In the release state of the gripping mechanism 50 illustrated in FIG. 16A, a minimum separation distance T between a distal end 80a of the protruding portion 80 and the first holding portion 31 is larger than the diameter of the string-like implant 60. Therefore, in the release state of the gripping mechanism 50 illustrated in FIG. 16A, the string-like implant 60 can be disposed in the holding space G2 of the gap G for forming the insertion passage 51 from the distal side to the proximal side in the axial direction A beyond the position of the protruding portion 80. In the release state of the gripping mechanism 50 illustrated in FIG. 16A, the implant 60 disposed in the holding space G2 is caught by the protruding portion 80, and thus the implant 60 is less likely to fall off from the holding space G2 to the distal side in the axial direction A. Further, in the gripping state of the gripping mechanism 50 illustrated in FIG. 16B, the protruding portion 80 overlaps the first holding portion 31 of the outer cylinder 23 in the radial direction C. Accordingly, in the gripping state of the gripping mechanism 50 illustrated in FIG. 16B, a distal side of the insertion passage 51 in the axial direction A is closed by the protruding portion 80. Therefore, in the gripping state of the gripping mechanism 50 illustrated in FIG. 16B, the implant 60 can be further prevented from unintentionally falling off from the distal side of the insertion passage 51.

A shape of the protruding portion 80 is not limited to a rectangular shape illustrated in FIGS. 16A and 16B. The protruding portion 80 may have, for example, a triangular shape that protrudes outward in the circumferential direction B from the outer edge portions 41a1 on both sides of the outer surface 41a in the circumferential direction B and gradually decreases in width in the axial direction A toward a distal end.

The protruding portion 80 is disposed closer to a distal side in the axial direction A than the holding space G2. However, a position in the axial direction A where the protruding portion 80 is disposed is preferably closer to a proximal side than the inlet space G1. That is, the protruding portion 80 is preferably disposed between the inlet space G1 and the holding space G2 in the axial direction A. Accordingly, it is possible to obtain the above-described effect of the protruding portion 80 while ensuring the effect of the inlet space G1 for guiding the implant 60 to the holding space G2.

As illustrated in FIG. 16A, when the gripping mechanism 50 is in the release state, the end surface 31a of the first holding portion 31, the outer edge portion 41a1 and the inner surface 41b of the second holding portion 41, which define the holding space G2 closer to the proximal side in the axial direction A than the protruding portion 80, extend substantially parallel to the axial direction A, but the disclosure is not limited to this configuration. The end surface 31a of the first holding portion 31, the outer edge portion 41a1 and the inner surface 41b of the second holding portion 41, which define the holding space G2, may be, for example, inclined relative to the axial direction A so as to approach each other from the proximal side toward the distal side in the axial direction A. The end surface 31a of the first holding portion 31, the outer edge portion 41a1 and the inner surface 41b of the second holding portion 41, which define the holding space G2, may be, for example, inclined relative to the axial direction A so as to be away from each other from the proximal side toward the distal side in the axial direction A.

Further, the end surface 31a of the first holding portion 31 and the outer edge portion 41a1 of the second holding portion 41, which hold the string-like implant 60, may be subjected to minute unevenness processing. In this manner, the string-like implant 60 can be prevented from sliding in the axial direction A.

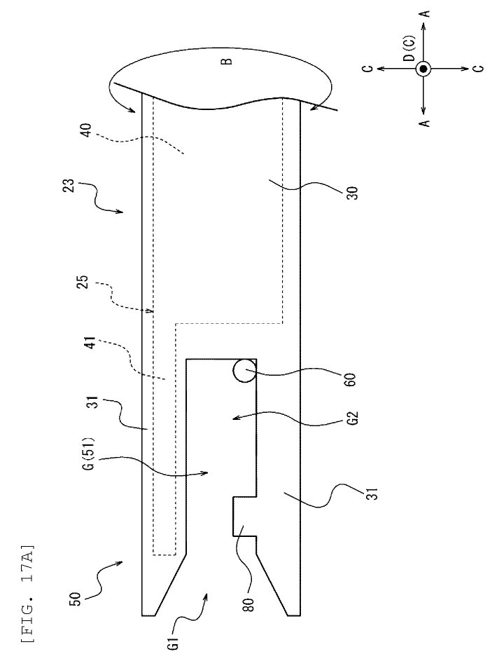

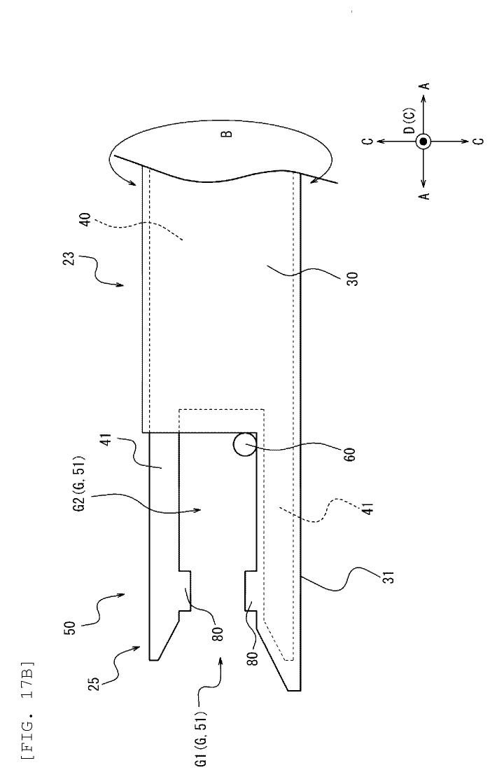

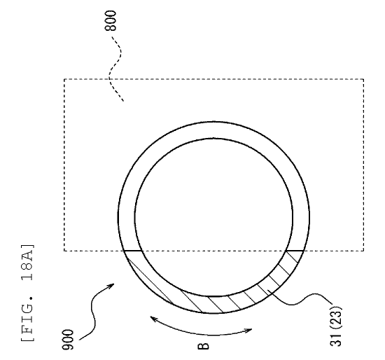

In FIGS. 16A and 16B, only the second holding portion 41 includes the protruding portion 80, but the disclosure is not limited to this configuration. For example, as illustrated in FIG. 17A, only the first holding portion 31 may include the protruding portion 80. For example, as illustrated in FIG. 17B, the first holding portion 31 and the second holding portion 41 may each include the protruding portion 80.

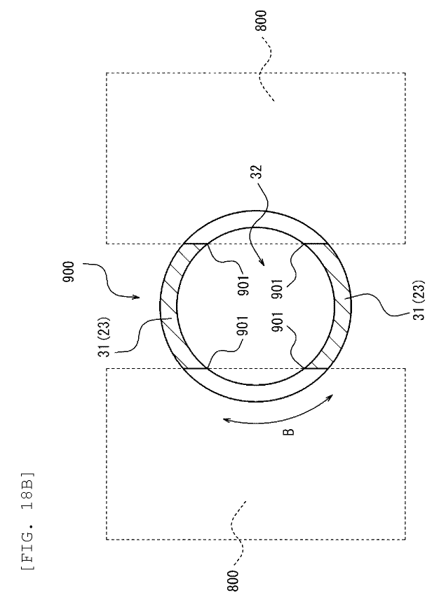

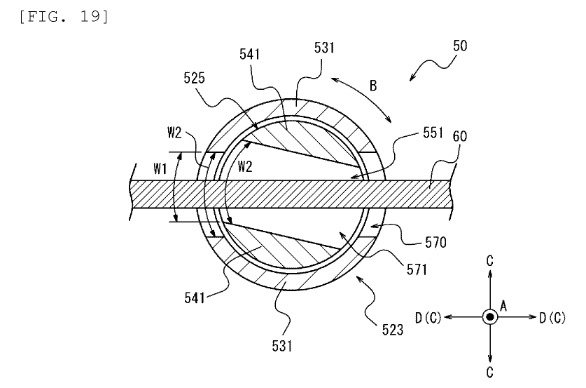

Finally, a manufacturing method for forming the first holding portion 31 will be described. First, a manufacturing method for forming only one first holding portion 31 provided in a partial region in the circumferential direction B will be described. As illustrated in FIG. 18A, the first holding portion 31 can be formed, for example, by cutting a part of a distal portion of a cylindrical body 900, which is a base of the outer cylinder 23, with a grindstone 800. With such a manufacturing method, only one first holding portion 31 can be easily formed. The same applies to a manufacturing method for forming only one second holding portion 41 (see FIG. 8A and the like) provided in a partial region of the shaft 25 in the circumferential direction B.

Next, a manufacturing method for forming two first holding portions 31 (see FIG. 11) provided at an interval in the circumferential direction B will be described. As illustrated in FIG. 18B, the two first holding portions 31 can be formed, for example, by cutting two different locations in the circumferential direction B of a distal portion of the cylindrical body 900, which is a base of the outer cylinder 23, with the grindstones 800. However, as illustrated in FIG. 18B, when the two first holding portions 31 are formed by the grindstones 800, edge portions 901 protruding toward the insertion groove 32 are likely to be formed at both ends of the first holding portions 31 in the circumferential direction B. Therefore, in such a case, it may be necessary to separately perform chamfering for removing the edge portions 901. The two first holding portions 31 may be formed by forming the insertion groove 32 by, for example, wire electric discharge machining, and laser cutting. In such a case, the above-described edge portions 901 protruding toward the insertion groove 32 are less likely to be formed, but may be expensive in terms of manufacturing cost. Based on the above, providing only one first holding portion 31 illustrated in FIG. 18A is advantageous from the viewpoint of manufacturing efficiency and manufacturing cost as compared with a case of providing two first holding portions 31 illustrated in FIG. 18B.

In a case where the shaft 25 is a columnar solid member, a manufacturing method for forming two second holding portions 41 of the shaft 25 (see FIG. 12) may be a manufacturing method for forming the insertion groove 42 (see FIG. 12) by, for example, wire electrical discharge machining, and laser cutting. When the shaft 25 is a cylindrical hollow member, the shaft 25 may be manufactured by the same method as that for the outer cylinder 23 described above.

A type of the string-like implant 60 illustrated in the above-described embodiment and modifications is not particularly limited, and examples thereof include collagen fibers, protein fibers, and biodegradable fibers. The implant 60 may be used to regenerate, for example, lymphatic vessels, blood vessels, and nerves.

The operation device and the implant indwelling instrument according to the present disclosure are not limited to the specific configurations illustrated in the above-described embodiments and modifications, and various modifications, changes, and combinations can be made without departing from the scope of the claims. Therefore, the operation device and the implant indwelling instrument implemented by appropriately combining the configurations illustrated in the above-described embodiments and modifications also belong to the technical scope of the present disclosure.

### Reference Character List

10: puncture needle

11: outer needle assembly

12: inner needle assembly

13: outer needle

13a: distal end of outer needle

13b: lumen of outer needle

14: inner needle

14a: distal end of inner needle

15: outer needle hub

16: inner needle hub

20: operation device

21: outer cylinder assembly

22: shaft assembly

23: outer cylinder

23a: lumen of outer cylinder

24: outer cylinder hub

25: shaft

26: shaft hub

30: outer cylinder main body portion

30a: distal end surface of outer cylinder main body portion

31: first holding portion

31a: end surface of first holding portion

32: insertion groove of outer cylinder

32a, 32b: groove wall of insertion groove of outer cylinder

40: shaft main body portion

40a: distal end surface of shaft main body portion

41: second holding portion

41a: outer surface of second holding portion

41a1: outer edge portion of outer surface of second holding portion

41b: inner surface of second holding portion

42: insertion groove of shaft

42a, 42b: groove wall of insertion groove of shaft

50: gripping mechanism

51: insertion passage

60: string-like implant

80: protruding portion

80a: distal end of protruding portion

100: implant indwelling instrument

523: outer cylinder

525: shaft

531: first holding portion

541: second holding portion

550: gripping mechanism

551: insertion passage

570: first Groove

571: second Groove

800: grindstone

900: cylindrical body

901: edge portion

A: axial direction of outer cylinder

B: circumferential direction of outer cylinder

C: radial direction of outer cylinder

D: penetration direction of gap

G: gap (insertion passage)

M: center line of gap

O: central axis of outer cylinder

T: minimum separation distance between distal end of protruding portion and first holding portion when gripping mechanism is in release state

W1: circumferential width at both ends of insertion passage

W2: circumferential width at both ends of first groove and second groove

W3: width of gap

BS: living body surface

What is claimed is:

1. An operation device comprising:
an outer cylinder that defines a lumen penetrating inside the outer cylinder in an axial direction; and
a shaft that is disposed in the lumen and is rotatable relative to the outer cylinder in a circumferential direction of the outer cylinder; wherein:
the outer cylinder comprises:
an outer cylinder main body portion having a cylindrical shape, and
one or more first holding portions that protrude from a distal end surface of the outer cylinder main body portion only in a partial region of the outer cylinder main body portion in the circumferential direction;
the shaft comprises:
a shaft main body portion having a cylindrical shape or a columnar shape, and
one or more second holding portions that protrude from a distal end surface of the shaft main body portion only in a partial region of the shaft main body portion in the circumferential direction;
an insertion passage is defined between the one or more first holding portions and the one or more second holding portions;
the one or more first holding portions and the one or more second holding portions are configured to hold an implant disposed in the insertion passage by rotating the shaft relative to the outer cylinder in the circumferential direction; and
a quantity of the one or more first holding portions of the outer cylinder is one, and/or a quantity of the one or more second holding portions of the shaft is one.

2. The operation device according to claim 1, wherein:
a quantity of the one or more first holding portions of the outer cylinder is one, and a quantity of the one or more second holding portions of the shaft is one.

3. The operation device according to claim 2, wherein:
the insertion passage is formed by a gap that is defined between the first holding portion and the second holding portion in a state in which the one first holding portion of the outer cylinder and the one second holding portion of the shaft are disposed to face each other in a radial direction of the outer cylinder, the gap having a distal end open to an outside; and
the gap includes an inlet space having a width gradually increasing toward the distal end.

4. The operation device according to claim 1, wherein:
a quantity of the one or more first holding portions of the outer cylinder is one, and a quantity of the one or more second holding portions of the shaft is two; and
an insertion groove through which the implant is inserted is defined between the two portions of the shaft.

5. The operation device according to claim 4, wherein:
the insertion passage is formed by a gap that is defined between the first holding portion and the second holding portion in a state in which the first holding portion of the outer cylinder and the second holding portion of the shaft are disposed to face each other in a radial direction of the outer cylinder, the gap having a distal end open to an outside; and
the gap or the insertion groove includes an inlet space having a width gradually increasing toward the distal end.

6. The operation device according to claim 1, wherein:
a quantity of the one or more first holding portions of the outer cylinder is two, and a quantity of the one or more second holding portions of the shaft is one; and
an insertion groove through which the implant is inserted is defined between the two holding portions of the outer cylinder.

7. The operation device according to claim 6, wherein:
the insertion passage is formed by a gap that is defined between the first holding portion and the second holding portion in a state in which the first holding portion of the outer cylinder and the second holding portion of the shaft are disposed to face each other in a radial direction of the outer cylinder, the gap having a distal end open to an outside; and
the gap or the insertion groove includes an inlet space having a width gradually increasing toward the distal end.

8. The operation device according to claim 1, wherein:
the insertion passage is formed by a gap that is defined between the one or more first holding portions and the one or more second holding portions in a state in which the one or more first holding portions of the outer cylinder and the one or more second holding portions of the shaft are disposed to face each other in a radial direction of the outer cylinder, the gap having a distal end open to an outside; and
the gap is eccentrically located outward in the radial direction relative to a central axis of the outer cylinder.

9. The operation device according to claim 2, wherein:
the insertion passage is formed by a gap that is defined between the one first holding portion and the one second holding portion in a state in which the one first holding portion of the outer cylinder and the one second holding portion of the shaft are disposed to face each other in a radial direction of the outer cylinder, the gap having a distal end open to an outside; and
the gap is eccentrically located outward in the radial direction relative to a central axis of the outer cylinder.

10. The operation device according to claim 3, wherein:
the insertion passage is formed by a gap that is defined between the one first holding portion and the one second holding portion in a state in which the one first holding portion of the outer cylinder and the one second holding portion of the shaft are disposed to face each other in a radial direction of the outer cylinder, the gap having a distal end open to an outside; and
the gap is eccentrically located outward in the radial direction relative to a central axis of the outer cylinder.

11. The operation device according to claim 4, wherein:
the insertion passage is formed by a gap that is defined between the one first holding portion and the two second holding portions in a state in which the one first holding portion of the outer cylinder and the two second holding portions of the shaft are disposed to face each other in a radial direction of the outer cylinder, the gap having a distal end open to an outside; and
the gap is eccentrically located outward in the radial direction relative to a central axis of the outer cylinder.

12. The operation device according to claim 6, wherein:
the insertion passage is formed by a gap that is defined between the two first holding portions and the one second holding portion in a state in which the two first holding portions of the outer cylinder and the one second holding portion of the shaft are disposed to face each other in a radial direction of the outer cylinder, the gap having a distal end open to an outside; and
the gap is eccentrically located outward in the radial direction relative to a central axis of the outer cylinder.

13. The operation device according to claim 1, wherein:
at least one holding portion of the one or more first holding portions of the outer cylinder and the one or more second holding portions of the shaft comprises a protruding portion protruding in the circumferential direction.

14. The operation device according to claim 2, wherein:
at least one holding portion of the one first holding portion of the outer cylinder and the one second holding portion of the shaft comprises a protruding portion protruding in the circumferential direction.

15. The operation device according to claim 3, wherein:
at least one holding portion of the one first holding portion of the outer cylinder and the one second holding portion of the shaft comprises a protruding portion protruding in the circumferential direction.

16. The operation device according to claim 4, wherein:
at least one holding portion of the one first holding portion of the outer cylinder and the two second holding portions of the shaft comprises a protruding portion protruding in the circumferential direction.

17. The operation device according to claim 6, wherein:
at least one holding portion of the two first holding portions of the outer cylinder and the one second holding portion of the shaft comprises a protruding portion protruding in the circumferential direction.

18. An implant indwelling instrument comprising:
an operation device comprising:
an outer cylinder that defines a lumen penetrating inside the outer cylinder in an axial direction, and
a shaft that is disposed in the lumen and is rotatable relative to the outer cylinder in a circumferential direction of the outer cylinder, wherein:
the outer cylinder comprises:
an outer cylinder main body portion having a cylindrical shape, and
one or more first holding portions that protrude from a distal end surface of the outer cylinder main body portion only in a partial region of the outer cylinder main body portion in the circumferential direction,
the shaft comprises:
a shaft main body portion having a cylindrical shape or a columnar shape, and
one or more second holding portions that protrude from a distal end surface of the shaft main body portion only in a partial region of the shaft main body portion in the circumferential direction,
an insertion passage is defined between the one or more first holding portions and the one or more second holding portions,
the one or more first holding portions and the one or more second holding portions are configured to hold an implant disposed in the insertion passage by rotating the shaft relative to the outer cylinder in the circumferential direction, and
a quantity of the one or more first holding portions of the outer cylinder is one, and/or a quantity of the one or more second holding portions of the shaft is one; and
a puncture needle including an outer needle into which the outer cylinder and the shaft of the operation device are insertable.

19. A method for indwelling an implant, the method comprising:
providing an operation device comprising:
an outer cylinder that defines a lumen penetrating inside the outer cylinder in an axial direction, and
a shaft that is disposed in the lumen and is rotatable relative to the outer cylinder in a circumferential direction of the outer cylinder, wherein:
the outer cylinder comprises:
an outer cylinder main body portion having a cylindrical shape, and
one or more first holding portions that protrude from a distal end surface of the outer cylinder main body portion only in a partial region of the outer cylinder main body portion in the circumferential direction,
the shaft comprises:
a shaft main body portion having a cylindrical shape or a columnar shape, and
one or more second holding portions that protrude from a distal end surface of the shaft main body portion only in a partial region of the shaft main body portion in the circumferential direction,
an insertion passage is defined between the one or more first holding portions and the one or more second holding portions, and
a quantity of the one or more first holding portions of the outer cylinder is one, and/or a quantity of the one or more second holding portions of the shaft is one;
holding an implant with the one or more first holding portions and the one or more second holding portions by rotating the shaft relative to the outer cylinder in the circumferential direction from a release state to a gripping state; and
inserting the outer cylinder and the shaft that hold the implant into an outer cylinder of a puncture needle;
conveying the implant to a target site in a living body; and
releasing the implant by rotating the shaft relative to the outer cylinder in the circumferential direction from the gripping state to the release state.
